(19) United States
(12) Patent Application Publication
(10) Pub. No.: US 2009/0004123 A1
(43) Pub. Date: Jan. 1, 2009

(54) Cosmetic Composition Comprising Polyorganosiloxane and Uses Thereof

(75) Inventors: Melissa Manuszak, Paris (FR); Gilles Lorentz, Lyon (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Rhodia Chimie, Boulogne Billancourt Cedex (FR)

(21) Appl. No.: 11/630,724

(22) PCT Filed: Jun. 15, 2005
(86) PCT No.: PCT/FR05/01489

§ 371 Date: Jun. 11, 2008

(30) Foreign Application Priority Data
Jun. 23, 2004 (FR) 04 06816

(51) Int. Cl. A61K 8/89

(52) U.S. Cl. 424/59; 424/70.122

(57) ABSTRACT

The invention relates to a cosmetic composition comprising a cosmetically acceptable vector and polyorganosiloxane. The polyorganosiloxane comprises a group that includes a sterically hindered amine, and a polyalkoxyl group.

The present invention relates to a cosmetic composition comprising a polyorganosiloxane and a cosmetically acceptable carrier.

A great many cosmetic compositions contain polyorganosiloxanes (“silicones”). Polyorganosiloxanes can be used for providing a conditioning effect of the hair or of the skin. Polyorganosiloxanes can also be used for sensory effects, called cosmetic effects, when applied to the skin, the hair or the lips.

Thus, polyorganosiloxanes of a great many different chemical structures, which can optionally bear different functional groups, have been proposed for use in cosmetic compositions.

Linear polydimethylorganosiloxanes (PDMS) can be used as sensory agents on the skin, as protective agents forming a barrier to water, as defoaming agents, as agents for removing creamy whiteness from cosmetic compositions appearing when first rubbed onto the skin or the hair (desoapers), as conditioners or as emollients.

Polyorganosiloxanes comprising polyether functions result from the replacement of methyl groups, on the siloxy backbone, with polyether (polyalkoxylated) substituents, in general ethylene oxide and propylene oxide substituents. This substitution causes a modification of the hydrophilicity/hydrophobicity balance (HLB) of the polyorganosiloxane. The addition of polyether groups allows a modification of the compatibility of the polyorganosiloxane with polar or nonpolar solvents. Thus, these polyorganosiloxanes comprising polyether functions may be water-soluble to soluble in oleophilic solvents. They may find uses in aqueous phases and in oily phases of cosmetic compositions. They may be used as emulsifiers of water-in-oil or water-in-cyclopentasiloxane emulsions. They may reinforce sensory effects in shower gel compositions. They may play a role as modifiers of hair spray resins. They may be used as gentle conditioners in transparent compositions.

Cosmetic compositions have also been described containing laminated polyorganosiloxanes, often with advantages in terms of coloration. The presence of amine groups improves the affinity of the polyorganosiloxane for the hair and provides good conditioning of the hair, for compositions that are intended to be rinsed or not to be rinsed.

Documents U.S. Pat. No. 6,605,577 (Chemsil Silicones Inc.), U.S. Pat. No. 6,642,194 (Chemsil Silicones Inc.), WO 03/088939 (The Procter & Gamble Company), WO 03/066007 (Dow Corning), describe cosmetic compositions comprising a polyorganosiloxane bearing particular functional groups.

There is a need for new compositions, offering the consumer added, improved or simplified functionality, and/or offering the manufacturer advantages in terms of ease of manufacture, versatility of formulation, compatibility between ingredients, and/or costs.

The present invention meets this need by offering novel cosmetic compositions.

Thus, the invention relates to a cosmetic composition comprising a cosmetically acceptable carrier and a polyorganosiloxane, characterized in that the polyorganosiloxane has the following general formula (I):

[RₐX_{b}Y_{c}SiO_{(4-a-b-c)/2}]_{N} (I)

where:
RₐX_{b}Y_{c}SiO_{(4-a-b-c)/2}, which may be identical or different, represent linear or branched units in the polyorganosiloxane,
N represents the number of silicon atoms in the polyorganosiloxane, greater than or equal to 3,
a, b and c, which may be identical or different, are numbers equal to 0, 1, 2 or 3,
a+b+c, which may be identical or different, is equal to 0, 1, 2 or 3,
R, which may be identical or different, represents a C₁-C₁₈ alkyl group, a C₆-C₁₂ aryl group, a C₆-C₁₂ aralkyl group, a C₆-C₁₂ alkaryl group, a group of formula —[CH₂]₃—NH[CH₂]₂—NH₂, a group of formula —[CH₂]₃—NH₂, a hydroxyl group, a C₁-C₁₈ alkoxy group, a C₁-C₁₈ hydroxyalkyl group, or a C₁-C₁₈ hydroxyalkyletheralkyl group, said groups being optionally substituted,
X, which may be identical or different, represents a polyalkoxy group carried by a silicon atom,
Y, which may be identical or different, represents a group carried by a silicon atom, of formula —R⁴—U-Hals, in which:
   R⁴ is a divalent hydrocarbon binding group, preferably an alkyl group,
   U is a covalent bond or a divalent swivel group comprising a heteroatom,
   Hals is a functional group comprising a sterically hindered amine,
at least one silicon atom of the polyorganosiloxane bears a group X, and
at least one silicon atom of the polyorganosiloxane bears a group Y.

The polyorganosiloxane of formula (I) may notably display improved formulability relative to other polyorganosiloxanes. In addition it can modulate, improve or provide interesting properties in terms of:
conditioning,
sensory effects,
prevention of aging of the skin and of the hair,
   appearance of the compositions, notably little yellowing, and/or
   other properties mentioned later in the present description.

The composition can include, in addition to the polyorganosiloxane of formula (I), ingredients selected from the following:
at least one anionic and/or amphoteric surfactant, alone or mixed,
optionally, at least one stabilizer and/or conditioner and/or conditioning aid, or a mixture of such agents,
optionally another polyorganosiloxane,
optionally a UV filter.

In a first section, details will be given of some of the ingredients that can be included in the cosmetic composition.

### Polyorganosiloxane of Formula (I)

The polyorganosiloxane has the following general formula (I):

[RₐX_{b}Y_{c}SiO_{(4-a-b-c)/2}]_{N} (I)

where:
RₐX_{b}Y_{c}SiO_{(4-a-b-c)/2}, which may be identical or different, represent linear or branched units in the polyorganosiloxane,
N represents the number of silicon atoms in the polyorganosiloxane, greater than or equal to 3,
a, b and c, which may be identical or different, are numbers equal to 0, 1, 2 or 3,
a+b+c, which may be identical or different, is equal to 0, 1, 2 or 3,
R, which may be identical or different, represents a C₁-C₁₈ alkyl group, a C₆-C₁₂ aryl group, a C₆-C₁₂ aralkyl group, a C₆-C₁₂ alkaryl group, a group of formula —[CH₂]₃—NH[CH₂]₂—NH₂, a group of formula —[CH₂]₃—NH₂, a hydroxyl group, a C₁-C₁₈ alkoxy group, a C₁-C₁₈ hydroxyalkyl group, or a C₁-C₁₈ hydroxyalkyletheralkyl group, said groups being optionally substituted,
X, which may be identical or different, represents a polyalkoxy group carried by a silicon atom,
Y, which may be identical or different, represents a group carried by a silicon atom, of formula —R⁴—U-Hals, in which:
   R⁴ is a divalent hydrocarbon binding group, preferably an alkyl group,
   U is a covalent bond or a divalent swivel group comprising a heteroatom,
   Hals is a functional group comprising a sterically hindered amine, and
at least one silicon atom of the polyorganosiloxane bears a group X, and
at least one silicon atom of the polyorganosiloxane bears a group Y.

In formula (I), the groups X and Y are preferably carried by different silicon atoms.

The group X advantageously has the formula L^{x}-Z^{x}-Palk, in which:
L^{x} is a divalent bonding group, preferably an alkyl group,
Z^{x} is a covalent bond or a divalent connecting group comprising a heteroatom,
Palk is a group of formula [OE]ₛ-[OP]ₜ—X′, in which
   OE is a group of formula —CH₂—CH₂—O—,
   OP is a group of formula —CH₂—CHCH₃—O or —CHCH₃—CH₂—O—,
   X′ is a hydrogen atom or a hydrocarbon-based group,
   s is an average number greater than 1, and
   t is an average number greater than or equal to 0.

The group X is preferably a group of formula —CH₂—CH₂—CH₂—O-[OE]_{q}-[OP]ᵣ—X′,

in which:
q≧5,
r≧0, and
X′ is a hydrogen atom.

Advantageously, group Y has the following formula (II):

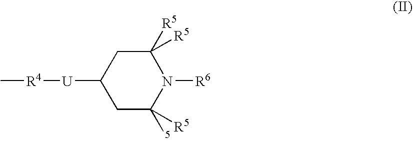

in which:
R⁴, which may be identical or different, is a divalent hydrocarbon divalent selected from:
   linear or branched alkylene groups having 2 to 18 carbon atoms;
   alkylene-carbonyl groups, the linear or branched alkylene portion of which has 2 to 20 carbon atoms;
   alkylene-cyclohexylene groups, the linear or branched alkylene portion of which has 2 to 12 carbon atoms and the cyclohexylene portion has an —OH group and optionally 1 or 2 alkyl groups having 1 to 4 carbon atoms;
   groups of formula —R⁷—O—R⁷ in which the R⁷ groups, which may be identical or different, represent alkylene groups having 1 to 12 carbon atoms;
   groups of formula R⁷—O—R⁷ in which the R⁷ groups have the meanings stated previously and one of them or both of them are substituted with one or two —OH groups;
   groups of formula —R⁷—COO—R⁷ in which the R⁷ groups have the meanings stated previously; and
   groups of formula —R⁸—O—R⁹—O—CO—R⁸ in which the R⁸ and R⁹ groups, which may be identical or different, represent alkylene groups having 2 to 12 carbon atoms and the R⁹ group is optionally substituted with a hydroxyl group;
U represents —O— or —NR¹⁰—, R¹⁰ being a hydrogen atom, a linear or branched alkyl group comprising 1 to 6 carbon atoms;
R⁵, which may be identical or different, is a linear or branched alkyl group having 1 to 3 carbon atoms or a phenyl group; and
R⁶ represents a hydrogen atom, an R⁵ group or a free radical O.

Group Y is preferably a group with the following formula (III):

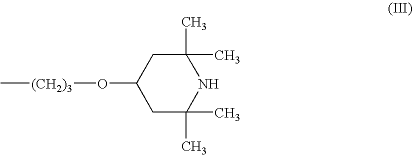

Group R, which may be identical or different, is advantageously a methyl, ethyl, isopropyl, tert-butyl, n-hexyl, octyl, trifluoropropyl, or phenyl group. Group R is preferably a methyl group.

The polyorganosiloxane of formula (I) is advantageously a linear polyorganosiloxane. Advantageously, it has the following formula (IV):

A-SiR₂—O—[SiRXO]ₓ—[SiRYO]ₙ—[SiR₂O]ₘ—O—SiR₂-A (IV)

in which:
R, which may be identical or different, is a group as described above,
X, which may be identical or different, is a group as described above,
Y, which may be identical or different, is a group as described above,
A, which may be identical or different, is a group R, Y, or X,
x is an average number greater than 0,
n is an average number greater than 0,
m is an average number greater than 0, and
x+m+n+2=N.

Preferably:
A is a group R, and
R is a methyl group.

Preferably:
m≧10, preferably m≧100
x≧1, and
n≧1, even more preferably n≧3.

The polyorganosiloxane of formula (I) advantageously comprises between 0.1 and 0.5 wt. % of nitrogen atoms, relative to the weight of polyorganosiloxane. This proportion can be calculated from the measured amount, in moles, of acid required for neutralizing the polyorganosiloxane.

Preferably:
m≧50, and
0.001≦n/(m+n+x)≦0.5, preferably 0.005≦n/(m+n+x)≦0.1.

Even more preferably:
m≧50, and
0.001≦x/(m+n+x)≦0.5, preferably 0.01≦x/(m+n+x)≦0.2.

Advantageously, the polyorganosiloxane of formula (I) has a viscosity between 1000 and 1000000 mPa·s, preferably between 5000 and 500000 mPa·s, preferably between 10000 and 100000 mPa·s. The viscosity can be measured at a temperature of 25° C. using a viscometer of the Brookfield type, for example at shear corresponding to 10 rev/min, spindle 4.

The polyorganosiloxane of formula (I) can be prepared by any suitable method. Methods that can be used are described in U.S. Pat. No. 5,721,297. We may notably mention methods comprising grafting of group Y by a hydrosilylation reaction. We may also mention methods of redistribution or rearrangement starting from polyorganosiloxanes not comprising the group Y, and/or polyorganosiloxanes comprising the group X, and polyorganosiloxanes comprising groups Y attached to silicon atoms.

For example, a suitable method of redistribution or rearrangement can comprise the following stages:

1. Bring into contact a polydimethylsiloxane, preferably cyclic, hexamethyldisiloxane, a copolymer of dimethylsiloxane and methylpoly(alkylene oxide), and a cyclic polymethylsiloxane comprising groups Y attached to silicon atoms.
2. Heat, introduce a basic catalyst, for example a strong base, and leave to react.
3. Neutralize, for example with a weak acid, then isolate a polyorganosiloxane of formula (I), for example by devolatilization, cooling and drawing off.

Note that the polyorganosiloxane of formula (I) can in fact be a complex composition comprising, in addition to the polyorganosiloxane of formula (I), varying amounts of compounds used for its preparation or by-products. In addition, the polyorganosiloxane of formula (I) may comprise a varying amount of free polyoxyalkylene, for example polyethylene oxide, and/or polypropylene oxide, and/or of polyethylene oxide/polypropylene oxide copolymer.

### Cosmetically Acceptable Carrier

Any cosmetically acceptable carrier permitting formulation of the polyorganosiloxane of formula (I), and production of the desired cosmetic composition, for the intended use, can be used. Different cosmetically acceptable carriers for different types of formulations are known to a person skilled in the art.

The following may be mentioned as examples of cosmetically acceptable carriers: aqueous carriers (containing water), alcoholic carriers (containing an alcohol, for example ethanol, isopropanol, ethylene glycol or polyethylene glycols), propylene glycol, aqueous-alcoholic carriers (comprising a mixture of water and an alcohol for example ethanol, isopropanol, ethylene glycol or polyethylene glycols). Certain oils, volatile or non-volatile, can also be used. We may mention for example the fluid silicones, such as cyclopentasiloxane, for example Mirasil CM5 marketed by Rhodia.

A person skilled in the art is able to select carriers that are suitable for the desired types of formulations, and for the intended uses. For example, aqueous carriers are generally used for shampoos or shower gels. A propylene glycol carrier can be used for compositions in the form of creams. A cyclomethicone carrier can be used for make-up compositions, for example for foundations.

### Surfactants

The composition can contain at least one surfactant. It can be a mixture of various surfactants. Preferably it is at least one anionic surfactant, alone or mixed. The surfactant can moreover comprise anionic surfactants, amphoteric surfactants (true amphoteric or zwitterionic), neutral surfactants and/or cationic surfactants, alone or mixed. Compositions comprising at least one amphoteric surfactant and optionally an anionic surfactant are particularly advantageous, notably for reasons of softness. The total content of surfactant in the composition can be between 5 and 30 wt. %.

For compositions intended for treatment of the hair, such as shampoos, the content of surfactant is advantageously between 10 and 20 wt. %. Such compositions can contain salts, for example sodium chloride or ammonium chloride, advantageously at a content below 3 wt. %.

For compositions intended for treatment of the skin, such as shower gels, the content of surfactant is advantageously between 5 and 15 wt. %. Such compositions also preferably contain at least 2 wt. % of salts, for example sodium chloride or ammonium chloride.

The proportion by weight of anionic surfactant relative to total surfactants is preferably above 50%, preferably above 70%.

The anionic surfactants can be selected from the following surfactants:
alkyl ester sulfonates, for example of formula R—CH(SO₃M)-CH₂COOR′, or the alkyl ester sulfates, for example of formula R—CH(OSO₃M)-CH₂COOR′, where R represents a C₈-C₂₀ alkyl radical, preferably C₁₀-C₁₆, R′ represents a C₁-C₆, preferably C₁-C₃, alkyl radical, and M represents an alkaline-earth cation, for example sodium, or the ammonium cation. We may mention quite particularly the methyl ester sulfonates with a C₁₄-C₁₆ radical R;
alkylbenzene sulfonates, more particularly C₉-C₂₀, primary or secondary alkyl sulfonates, notably C₈-C₂₂, the alkyl glycerol sulfonates;
alkyl sulfates for example of formula ROSO₃M, where R represents a C₁₀-C₂₄, preferably C₁₂-C₂₀, alkyl or hydroxyalkyl radical; M denotes a cation with the same definition as above;
alkyl ether sulfates for example of formula RO(OA)ₙSO₃M, where R represents a C₁₀-C₂₄, preferably C₁₂-C₂₀ alkyl or hydroxyalkyl radical; OA represents an ethoxylated and/or propoxylated group; M represents a cation with the same definition as above, and n generally varies from 1 to 4, for example lauryl ether sulfate with n=2;
alkyl amide sulfates, for example of formula RCONHR′OSO₃M, where R represents a C₂-C₂₂, preferably C₆-C₂₀ alkyl radical, R′ represents a C₂-C₃ alkyl radical, M represents a cation of the same definition as above, as well as their polyalkoxylated (ethoxylated and/or propoxylated) derivatives (alkylamidoether sulfates);
salts of saturated or unsaturated fatty acids, for example those with C₈-C₂₄, preferably C₁₄-C₂₀ and an alkaline-earth cation, N-acyl N-alkyl taurates, alkyl isethionates, alkyl succinamates and alkylsulfo-succinates, monoesters or diesters of sulfosuccinates, N-acyl sarcosinates, polyethoxy carboxylates
mono- and di-ester phosphates, for example of the following formula: (RO)ₓ—P(═O) (OM)ₓ or R represents an alkyl, alkaryl, aralkyl, aryl radical, optionally polyalkoxylated, x and x′ being equal to 1 or 2, provided that the sum of x and x′ is equal to 3, M representing an alkaline-earth cation.

The non-ionic surfactants can be selected from the following surfactants:
alkoxylated aliphatic alcohols
alkoxylated triglycerides
alkoxylated fatty acids
alkoxylated sorbitan esters
alkoxylated aliphatic amines
alkoxylated di(phenyl-1 ethyl)phenols
alkoxylated tri(phenyl-1 ethyl)phenols
alkoxylated alkyl phenols
products resulting from the condensation of ethylene oxide with a hydrophobic compound resulting from the condensation of propylene oxide with propylene glycol, such as the Pluronics marketed by BASF;
products resulting from the condensation of ethylene oxide the compound resulting from the condensation of propylene oxide with ethylenediamine, such as the Tetronics marketed by BASF;
alkylpolyglycosides such as those described in U.S. Pat. No. 4,565,647;
amides of, e.g. C₈-C₂₀, fatty acids.

The amphoteric surfactants (true amphoteric comprising an ionic group and a potentially ionic group of opposite charge, or zwitterionic comprising two opposite charges simultaneously) can be selected from the following surfactants:
betaines in general, notably carboxybetaines of for example lauryl betaine (Mirataine BB from the company Rhodia) or octylbetaine; amidoalkyl betaines, such as cocamidopropyl betaine (CAPB) (Mirataine BDJ from the company Rhodia Chimie);
sulfo-betaines or sultaines such as cocamidopropyl hydroxy sultaine (Mirataine CBS from Rhodia);
alkylamphoacetates and alkylamphodiacetates, for example comprising a coco or lauryl chain (Miranol C2M, C32, L32 notably, from Rhodia);
alkylamphopropionates or alkylamphodipropionates (Miranol C2M SF);
alkyl amphohydroxypropyl sultaines (Miranol CS).

The cationic surfactants can be selected from the primary, secondary or tertiary aliphatic amine salts, optionally polyethoxylated, quaternary ammonium salts such as the chlorides or bromides of tetraalkylammonium, alkylamidoalkylammonium, trialkylbenzylammonium, trialkylhydroxyalkylammonium, or of alkylpyridinium, imidazoline derivatives, oxides of amines with cationic character.

Stabilizer and/or Conditioner and/or Conditioning Aid

The cosmetic composition according to the invention can advantageously comprise at least one stabilizer and/or conditioner (conditioners) and/or conditioning aid. They are also sometimes called suspending agents. “Conditioning aid” means that the presence of the agent improves the conditioning associated with other compounds, for example oils or silicones. The agents are understood as being agents different from the polyorganosiloxane of formula (I). Such agents are known to a person skilled in the art. The composition according to the invention can comprise several of these agents (mixtures or combinations), for combining their effects and/or creating synergies. Furthermore, some agents can have several functions. This applies for example to the polysaccharides, and their cationic derivatives, for example cationic derivatives of guars.

The proportion by weight of such agents can typically be from 0.1 to 10 wt. %, preferably from 0.3 to 8 wt. %, for polysaccharides or other agents.

As examples of stabilizers, we may mention:
crosslinked polyacrylates, for example polymers of the CARBOPOL or CARBOMER type marketed by BF Goodrich or Noveon, ACRITAMER marketed by RITA or TEGO CARBOMER marketed by Goldschmidt. These compounds can typically be present in an amount from 0.1 to 3 wt. %, preferably from 0.3 to 2 wt. % relative to the composition;
copolymers of acrylates/aminoacrylates/itaconates C₁₀-C₃₀ alkyl PEG 20 sold by National Starch under the name STRUCTURE PLUS. These compounds can typically be present in an amount from 0.1 to 3 wt. %, preferably from 0.3 to 2 wt. %, relative to the composition;
insoluble solids forming a network in the composition. These can be mono- and/or di-esters of fatty acids of ethylene glycol, the fatty acids preferably being C₁₆-C₁₈. It can in particular be ethylene glycol distearate (EGDS), for example marketed by Rhodia as a concentrate with other ingredients under the name MIRASHEEN. This compound can typically be present in an amount from 3 to 10 wt. %, preferably from 5 to 8 wt. %, relative to the composition.

We may also mention viscosity-increasing, gelling or texturizing agents such as the anionic acrylic copolymers of the ACULYNE type marketed by ISP or Rohm & Haas, the polysaccharides and their non-cationic derivatives such as cellulose derivatives such as hydroxypropylcellulose, carboxymethylcellulose, non-ionic derivatives of guars such as hydroxypropyl guar (for example Jaguar HP marketed by Rhodia), carob, tara gum or cassia gum, xanthan gum (for example Rhodicare sold by Rhodia), succinoglycans (for example Rheozan sold by Rhodia), alginates, carrageenans, chitin derivatives or any other polysaccharide with a texturizing function. These polysaccharides and their derivatives can be incorporated alone or in synergistic combination with other polysaccharides. These compounds can typically be present in an amount from 0.1 to 3 wt. %, preferably from 0.3 to 1 wt. %, relative to the composition.

We may mention, as examples of stabilizers and/or conditioners and/or conditioning aids:
cationic polymers derived from polysaccharides, for example cationic derivatives of celluloses, cationic derivatives of starches, cationic derivatives of guars, cationic derivatives of carob,
synthetic cationic polymers,
mixtures or combinations of these agents.

Cationic polymers, synthetic or otherwise, that can provide a function of conditioning agent are notably polymers of the polyquaternium type, for example polyquaternium-1, polyquaternium-2, polyquaternium-4, polyquaternium-5, polyquaternium-6 (also known as Merquat 1000 available from Nalco), polyquaternium-7 (also known as Merquat 5500 available from Nalco), polyquaternium-8, polyquaternium-9, polyquaternium-10 (also known as Polymer JR 400, sold by Amercol), polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22 (also known as Merquat 280, 281, 298 available from Nalco), polyquaternium-24, polyquaternium-27, polyquaternium-28, polyquaternium-29 (also known as Kytamer KCO available from Amercol), polyquaternium-30, polyquaternium-31, polyquaternium-32, polyquaternium-33, polyquaternium-34, polyquaternium-35, polyquaternium-36, polyquaternium-37, polyquaternium-39 (also known as Merquat 3300, 3331 available from Nalco), polyquaternium-44, polyquaternium-27 (also known as Merquat 2001 available from Nalco) and polyquaternium-55.

The cationic derivatives of the guars can have a function of stabilizer of the formulations, conditioner and/or conditioning aid. We may mention as examples:
guar hydroxypropyl trimonium chloride (Jaguar C13S, Jaguar C14S, Jaguar C17, Jaguar Excel, Jaguar C 2000, sold by RHODIA),
hydroxypropyl guar hydroxypropyl trimonium chloride (Jaguar C162, sold by RHODIA),
ether of poly(oxyethanediyl-1,2) hydroxy-2 chloride of trimethylammonium-3 propyl cellulose or polyquaternium-10.

### Other Polyorganosiloxane

The composition according to the invention can comprise a polyorganosiloxane different from the polyorganosiloxane of formula (I). This other polyorganosiloxane can be present for example in the form of an emulsion, defining a second family of droplets in the cosmetically acceptable carrier (often called co-emulsions). It can also be mixed with the polyorganosiloxane of formula (I), as an emulsion of droplets of the mixture (which can be called emulsions of mixtures), or as droplets dispersed in the polyorganosiloxane of formula (I) (which can be called multiple emulsions).

The polyorganosiloxane different from the polyorganosiloxane of formula (I) can be a polyorganosiloxane comprising polar groups, or a nonpolar polyorganosiloxane.

The following may be mentioned as examples of polyorganosiloxanes comprising polar groups: dimethiconols, amodimethicones, trimethylsilyl amodimethicone, dimethicone copolyols, ternary copolyols, Silatrizole, dimethicone copolyol amine, silicone quaternium (CTFA silicone quaternium 1 to 10).

We may mention, as examples of nonpolar polyorganosiloxanes, polydimethylorganosiloxanes (PDMS or dimethicone), silicones having phenyl groups, silsesquioxanes (structure “T”) and silicates (structure “Q”), crosslinked silicones, copolymers comprising silicone groups, silicone resins, silicone waxes, volatile alkyl methyl siloxanes.

Just as for the polyorganosiloxane of formula (I), the emulsions of polyorganosiloxane different from the polyorganosiloxane of formula (I) can be prepared by emulsification in situ or by prior emulsification, and can have droplet sizes less than 0.15 μm, or between 0.15 μm and 2 μm, or greater than or equal to 2 μm. Reference should be made to the passage dealing with emulsions hereafter.

### UV Filters

The composition according to the invention can comprise UV filtering agents. These can be organic or mineral agents. They can for example be mineral agents such as dispersions of particles based on titanium dioxide, zinc oxide, or cerium oxide, preferably in the form of nanoparticles, if necessary covered with a layer based on oxide or hydroxide of silicon or aluminium, for example the dispersion sold under the name Mirasun® TiW60 by Rhodia. They can also be organic molecules. Such molecules are known by a person skilled in the art. As examples of organic molecules, we may mention the following compounds: Eusolex OCR or Eusolex 6300 (Merck); Parsol 1789, Parsol HS, or Parsol MCX (Givaudan Roure); Mexoril SX (Chimex); Escalol 567, Escalol 587, or Escalol 507 (ISP/Van Dyk); Uvinul MS-40, Uvinul T-150, or Spectrasorb UV-24 (BASF); Neo Heliopan MA or Neo Heliopan Type E 1000 (Haarmann & Reimer); Tinosorb M (Ciba), homomethyl salicylate.

### Other Ingredients

The following may be mentioned as other ingredients that may be included in the composition: coloring agents, dyes or colorants, fragrances, perfumes, odour-masking agents, polymers, buffers, complexing agents, complexing capsules, soluble salts, for example salts of metals, of alkalies, alkaline-earths or ammonium salts, for example NaCl or NaSO₄ or NH₄Cl, Lewis acids, particular thickeners, polymeric thickeners, thickening waxes, oils, emollients, humectants, moisturizers, luster agents, opacifiers, dispersants, agents promoting the suspension of particles, antimicrobial agents, preservatives, proteins, vegetable extracts, oxidizing agents, agents for altering viscosity, gelling agents, chelating agents, reducing agents.

The composition can additionally comprise a wide variety of active agents, hydrophilic or non-hydrophilic. These can be antifungal agents, antibacterial agents, for example triclosan, anti-dandruff agents, for example zinc pyrithione, anti-aging agents, anti-cellulite agents.

As examples of active substances that can be used in the field of cosmetics, we may mention the vitamins, such as vitamin A and its derivatives notably its esters such as acetate, palmitate, propionate, vitamin B2, pantothenic acid, vitamin D and vitamin E; mono-, di- and triglycerides; bactericides; UV absorbers, such as aminobenzoate derivatives of the PABA and PARA type, salicylates, cinnamates, anthranilates, dibenzoyl methanes, derivatives of camphor and mixtures thereof.

Anti-aging agents can also be used. As examples of such agents we may notably mention retinoids, fat-soluble vitamins, derivatives of vitamin C such as esters and notably acetate, propionate, palmitate; ceramides, pseudo-ceramides, phospholipids, fatty acids, aliphatic alcohols, cholesterol, sterols and mixtures thereof. As preferred fatty acids and aliphatic alcohols, we may more particularly mention those possessing linear or branched alkyl chains containing from 12 to 20 carbon atoms. Linoleic acid may be mentioned in particular.

It is also possible to use anti-cellulite agents, such as notably isobutylmethylxanthine and theophylline; as well as anti-acne agents, for example resorcinol, resorcinol acetate, benzoyl peroxide and a great many natural compounds.

Aromas, perfumes, essential oils, and essences can also be used as active substance. We may mention as examples: oils and/or essences of mint, spearmint, peppermint, menthol, vanilla, cinnamon, laurel, anise, eucalyptus, thyme, sage, cedar leaf, nutmeg, citrus (lemon, lime, grapefruit, orange), fruits (apple, pear, peach, cherry, plum, strawberry, raspberry, apricot, pineapple, grape, etc.), alone or as mixtures. It is also possible to employ compounds such as benzaldehyde, isoamyl acetate, ethyl butyrate, etc.

Antimicrobial agents can be selected from thymol, menthol, triclosan, 4-hexylresorcinol, phenol, eucalyptol, benzoic acid, benzoic peroxide, butyl paraben, and mixtures thereof.

The cosmetic compositions can also contain polymers possessing film-forming properties, which can be used to provide a fixing function. These polymers are generally present at concentrations between 0.01 and 10%, preferably between 0.5 and 5%. They are preferably of the polyvinylpyrrolidone type, copolymers of polyvinylpyrrolidone and methyl methacrylate, copolymer of polyvinylpyrrolidone and vinyl acetate, ethylene glycol/polyethylene glycol polyterephthalate copolymers, sulfonated terephthalic copolyester polymers.

Moisturizers can also be incorporated in the cosmetic compositions. As illustration of these, we may notably mention glycerol, propylene glycol, urea, collagen, gelatin, and emollients which are generally selected from alkylmonoglycerides, alkyldiglycerides, triglycerides such as oils extracted from plants and vegetables or their hydrogenated derivatives, mineral oils or paraffin oils, diols, aliphatic esters, and silicones (see above).

Preservatives such as esters of p-hydroxybenzoic acid, sodium benzoate, or any chemical agent that prevents the proliferation of bacteria or molds and is used conventionally in cosmetic compositions, are generally added to these compositions at the rate of 0.01 to 3 wt. %. Preservatives are for example sold under the names Glydant, Germaben, Kathon.

### Physicochemical Form of the Cosmetic Composition

The cosmetic composition comprises a cosmetically acceptable carrier and the polyorganosiloxane of formula (I). The polyorganosiloxane is dispersed in the cosmetically acceptable carrier, or in a mixture of ingredients comprising the cosmetically acceptable carrier. The dispersion can be for example:
a solution of the polyorganosiloxane of formula (I) in the cosmetically acceptable carrier or in a mixture comprising the cosmetically acceptable carrier;
a stable emulsion of droplets comprising the polyorganosiloxane of formula (I) in the cosmetically acceptable carrier,
an inverse emulsion in which the polyorganosiloxane of formula (I) is included in the external phase, or
a combination of phases separated into at least one layer comprising the cosmetically acceptable carrier, and a layer comprising the polyorganosiloxane of formula (I), which can form a dispersion of droplets comprising the polyorganosiloxane of formula (I) in the cosmetically acceptable carrier after agitation by the user.

The cosmetically acceptable carrier can of course include other ingredients than the polyorganosiloxane of formula (I), and these other ingredients can be present in solution or in dispersion, for example in the form of a suspension of solid particles, or an emulsion defining a family of droplets.

Moreover, the polyorganosiloxane of formula (I) can define a phase in which one or more other ingredients are dispersed.

Some characteristics of cosmetic compositions according to the invention in the form of emulsions are described below.

### Emulsions

The cosmetic compositions according to the invention can be in the form of emulsions of droplets comprising the polyorganosiloxane of formula (I) dispersed in the cosmetically acceptable carrier, preferably in an aqueous carrier.

The droplets of the emulsion can be of variable size. We may thus refer to microemulsions, mini-emulsions or macroemulsions. In the present application, the term “emulsion” notably covers all these types of emulsions. Without wishing to be bound to a particular theory, it can be stated that the microemulsions are generally thermodynamically stable systems, generally comprising large amounts of emulsifiers. The other emulsions are generally systems in a non-thermodynamically stable state, conserving for a certain time, in a metastable state, the mechanical energy supplied during their emulsification. These systems generally comprise smaller amounts of emulsifiers.

The compositions in the form of emulsions can be obtained by mixing the carrier, preferably aqueous, the polyorganosiloxane of formula (I), and generally an emulsifier, followed by emulsification. This can be called emulsification in situ.

The compositions in the form of emulsion can also be obtained by mixing the carrier, preferably aqueous, with a previously prepared emulsion of droplets comprising the polyorganosiloxane of formula (I) in an external phase, preferably miscible with the cosmetically acceptable carrier, preferably of the same nature as said carrier, preferably an aqueous carrier. This embodiment may be preferred as it is simpler to carry out. Furthermore, this embodiment is particularly suitable for cosmetic compositions in which the polyorganosiloxane of formula (I) is in the form of a microemulsion. This can be called pre-emulsification.

According to a particular embodiment, the emulsion is a microemulsion, with droplet size less than 0.15 μm. In this embodiment, the composition preferably comprises a proportion greater than 10 wt. %, preferably at least 15 wt. % of emulsifier relative to the weight of polyorganosiloxane of formula (I).

The size of the microemulsion droplets can be measured on an emulsion prepared prior to its addition to the cosmetic composition, by quasi-elastic light scattering (QELS), for example as described below. The equipment used comprises for example a Spectra-Physics 2020 laser, a Brookhaven 2030 correlator and the associated software. As the sample is concentrated, it is diluted in deionized water and filtered at 0.22 μm, so that it is finally at 2 wt. %. The diameter obtained is an apparent diameter. The measurements are conducted at angles of 90° and 135°. For the size measurements, in addition to conventional analysis by cumulants, three applications of the autocorrelation function are used (exponential sampling or EXPSAM described by Prof. Pike, the non-negatively constrained least squares or NNLS method, and the CONTIN method described by Prof. Provencher), each of which gives a size distribution weighted by the scattered intensity, and not by mass or number. The refractive index and the viscosity of the water are taken into account.

According to an advantageous embodiment, the microemulsion is transparent. The microemulsion can for example have a transmittance of at least 90%, preferably of at least 95%, at a wavelength of 600 nm, measured for example using a Lambda 40 UV-Vis spectrometer, at a concentration of 0.5 wt. % in water. In this connection the cosmetic composition can advantageously be transparent. It can for example have a transmittance of at least 90%, preferably of at least 95%, at a wavelength of 600 nm, measured for example using a Lambda 40 UV-Vis spectrometer.

According to another particular embodiment, the emulsion is an emulsion in which the average droplet size is greater than or equal to 0.15 μm, for example greater than 0.5 μm, or greater than 1 μm, or greater than 2 μm, or greater than 10 μm, or greater than 20 μm, and preferably less than 100 μm. The size of the droplets can be measured on an emulsion prepared prior to its addition to the cosmetic composition, or directly on the cosmetic composition diluted in water, by optical microscopy and/or laser granulometry (Horiba LA-910 laser scattering analyser). In this embodiment, the composition preferably comprises a proportion of less than 10 wt. % of emulsifier, relative to the weight of polyorganosiloxane of formula (I).

As mentioned above, the droplets of the emulsion can comprise ingredients other than the polyorganosiloxane of formula (I). Thus, the polyorganosiloxane of formula (I) can be mixed with a miscible ingredient, for example an oil, preferably a silicone oil, the mixture forming an emulsion. The droplets of polyorganosiloxane of formula (I) can also comprise an emulsion of smaller droplets of an immiscible phase (internal phase). The emulsion is then a multiple emulsion comprising an internal phase dispersed in an intermediate phase comprising the polyorganosiloxane of formula (I), itself dispersed in the carrier. The ingredients that can be comprised in the internal phase can be for example active ingredients producing a positive effect on the skin and/or the hair. They may also be agents promoting deposition of the polyorganosiloxane of formula (I), or of other ingredients, on the skin and/or the hair.

### Emulsifiers

The emulsifiers are agents that can make it possible to obtain an emulsion of the polyorganosiloxane of formula (I) in the carrier, preferably water. For example it may be:
a non-ionic surfactant,
a non-ionic, amphiphilic polymer, optionally combined with one or more anionic surfactants and/or anionic amphiphilic polymers,
a particular surfactant optionally combined with a co-surfactant, or
a protective colloid.

Particular Surfactant

According to a particular embodiment, the emulsifier is a particular surfactant optionally combined with a co-surfactant.

The particular surfactant is preferably selected from particular solids, with contact angle close to 0°, combined with at least one co-stabilizer selected from the non-ionic, anionic, cationic or zwitterionic surfactants.

The particular surfactant is for example a precipitated silica, a colloidal silica, an aluminosilicate, zinc oxide, titanium oxide, or a mixture of these compounds, these compounds comprising a surface treatment if necessary.

Protective Colloid

According to another particular embodiment the emulsifier is a protective colloid. It may for example be a polyvinyl alcohol, partially hydrolyzed if necessary.

The content of protective colloid is advantageously from 3 to 30 wt. % dry, relative to the internal emulsion, and preferably from 5 to 25%.

Non-Ionic Surfactant

According to another particular embodiment, the emulsifier comprises a non-ionic surfactant. It is preferably a non-ionic polyalkoxylated surfactant, selected for example from:
alkoxylated aliphatic alcohols
alkoxylated triglycerides
alkoxylated fatty acids
alkoxylated sorbitan esters
alkoxylated aliphatic amines
alkoxylated di(phenyl-1 ethyl)phenols
alkoxylated tri(phenyl-1 ethyl)phenols, and
alkoxylated alkyl phenols
   where the number of alkoxy units, more particularly oxyethylene and/or oxypropylene, is such that the HLB value is greater than or equal to 10.

Non-Ionic Amphiphilic Polymer

According to another particular embodiment, the external emulsifier comprises a non-ionic amphiphilic polymer. This polymer can be combined with one or more anionic surfactants and/or anionic amphiphilic polymers.

As an example of non-ionic amphiphilic polymers we may mention the triblock copolymers (polyethylene glycol)-(polypropylene glycol)-(polyethylene glycol).

With regard to non-ionic or anionic amphiphilic polymers, it is possible to employ a polymer comprising at least two blocks, one hydrophilic and the other hydrophobic. It is possible to use a comb copolymer.

Said amphiphilic polymers can be obtained, advantageously, by so-called living or controlled radical polymerization. As non-limiting examples of methods of so-called living or controlled polymerization, we may notably refer to applications WO 98/58974, WO 00/75207 and WO 01/42312 (xanthate), WO 98/01478 (dithioesters), WO 99/03894 (nitroxides); WO 99/31144 (dithiocarbamates), WO 02/26836 (dithiocarbazates); WO 02/10223 (dithiophosphoroesters), WO 96/30421 (atom transfer radical polymerization—ATRP).

The amphiphilic polymers can also be obtained by anionic polymerization.

They can also be prepared by means of (notably anionic) ring opening polymerizations, or by chemical modification of the polymer.

More particularly, regarding the non-ionic amphiphilic polymer, preferably polyoxyalkylenated, present in the external aqueous phase, it can be selected from polymers that are miscible at least partially in the external aqueous phase and preferably from the polyethylene glycol-polypropylene glycol-polyethylene glycol triblock copolymers.

It should be noted that polymers of the polyvinyl alcohol type or poly(acrylic acid)/poly(butyl acrylate)/poly(acrylic acid) triblock copolymers can be used for this purpose.

### Type of Formulation of the Composition and Uses

The composition according to the invention can be formulated in various forms, depending on the appearance it is desired to have, sensory properties (viscosity, touch, permanence, etc.) it is desired to have, and of course its intended use. The different types of formulations and the various uses are modulated by the nature and amount of the ingredients in the composition, and are familiar to a person skilled in the art.

Thus, the composition can be formulated as gels, fluids of different viscosities, milks, creams, oils, sprays, mousses, stick gels, pastes, lotions, dye concentrates etc.

The compositions can notably be selected from the compositions listed in Table I below, with physicochemical forms of the polyorganosiloxane of formula (I), types of formulation and uses also listed in Table (I) below. For these compositions, physicochemical forms, type of formulations and uses, reference may be made to the more detailed sections of the present application.

TABLE I

| | Physico-chemical | | |
|---|---|---|---|
| | form of the | | |
| | polyorganosiloxane | Type of | |
| Composition | of formula (I) | formulation | Use |
| Shampoos | Emulsion | Fluid | Cleaning |
| | | | and/or care |
| | | | of the hair |
| | | | and/or |
| | | | temporary |
| | | | coloring |
| | | | and/or |
| | | | fixing of |
| | | | coloring, |
| | | | with rinsing |
| After- | Emulsion | Fluid | Hair care, |
| shampoo | | | and/or |
| | | | untangling |
| | | | and/or |
| | | | hairdressing |
| | | | aid and/or |
| | | | temporary |
| | | | coloring |
| | | | and/or |
| | | | fixing of |
| | | | coloring |
| | | | and/or |
| | | | conditioning |
| | | | and/or |
| | | | conditioning |
| | | | after |
| | | | coloring, |
| | | | with or |
| | | | without |
| | | | rinsing |
| Shower gel | Emulsion | Fluid or | Cleaning |
| | | gel | and/or care |
| | | | of the skin |
| Hair mask | Emulsion | Very | Hair care |
| | | viscous | |
| | | fluid | |
| Sun cream | Emulsion | Cream | UV |
| | | | protection |
| | | | of the skin |
| Sun milk | Emulsion | Milk | UV |
| | | | protection |
| | | | of the skin |
| Sun oil | Inverse emulsion | Oil | UV |
| | or solution | | protection |
| | | | of the skin |
| Sun spray | Emulsion | Fluid | UV |
| | | | protection |
| | | | of the skin |
| Care cream | Emulsion | Cream | Skin care |
| Make-up | Emulsion | Cream or | Care and/or |
| remover | | fluid or | cleaning of |
| | | gel | the skin |
| | | | and/or of |
| | | | the |
| | | | eyelashes |
| Make-up | Emulsion, or | Cream, | Coloring of |
| | inverse suspension | fluid, | the skin or |
| | or solution | mascara, | of the |
| | | powder, | eyelashes |
| | | gels, | |
| | | sticks | |
| Deodorant | Emulsion or | Aerosol, | Reduction of |
| | inverse emulsion | gel, | the effects |
| | | sticks, | of sweating, |
| | | substance | applied on |
| | | that can be | the skin |
| | | applied | |
| | | using a | |
| | | ball | |
| | | applicator | |
| Shaving foam | Emulsion | Very fluid | Preparation |
| | | liquid or | for shaving |
| | | gel forming | |
| | | a foam | |
| | | after | |
| | | aerosol | |
| | | spraying | |
| Hairdressing | Emulsion | Fluid | Shaping of |
| or fixing | | | the hair |
| spray | | | |
| Hairdressing | Emulsion | Gel | Shaping of |
| or fixing | | | the hair |
| gel | | | |
| Hairdressing | Emulsion | Very fluid | Shaping of |
| or fixing | | liquid or | the hair |
| mousse | | gel forming | |
| | | a foam | |
| | | after | |
| | | aerosol | |
| | | spraying | |
| Dyeing | Emulsion | Gel or | Permanent or |
| composition | | viscous | semi- |
| | | liquid | permanent |
| | | | dyeing |

Among the uses of the compositions, we may mention uses in which the composition is intended to be rinsed and uses in which the composition is intended not to be rinsed.

### Compositions Intended to be Rinsed (“Rinse-Off Compositions”)

According to interesting embodiments, the composition is a composition for care of the skin and/or of the hair, preferably for cleaning and/or treatment of the skin and/or of the hair, said composition being in the form of a fluid. It is advantageously a shower gel, a shampoo, a rinse-off after-shampoo, a skin or hair mask that is intended to be rinsed after use.

For shower gels and shampoos, the composition can advantageously comprise:
at least one anionic and/or amphoteric surfactant, alone or mixed,
optionally, at least one stabilizer and/or conditioner and/or conditioning aid, or a mixture of such agents,
optionally another polyorganosiloxane,
mixtures of these ingredients.

Such ingredients have been described above.

For after-shampoos that are intended to be rinsed, the composition can advantageously be a quite viscous formulation, for example a cream, in the form of an emulsion comprising an aqueous phase in which a texturizing oily emulsified phase and emulsified droplets of the polyorganosiloxane of formula (I) are dispersed. The aqueous phase advantageously comprises a conditioner, for example a cationic polymer. Such polymers have been described above. The aqueous phase can also advantageously comprise a cationic surfactant. Such surfactants have been described above. It may for example be stearyl benzyl dimethyl ammonium chloride, cetyl trimethyl ammonium chloride (cetrimonium chloride), distearyl dimethyl ammonium chloride or stearamidopropyldimethylamine, for example in an amount from 0.3 to 2 wt. %.

### Compositions Intended not to be Rinsed (“Leave-on Compositions”)

According to interesting embodiments, the composition is a composition for the care of the skin and/or of the hair, in the form of a fluid or in some other form, preferably for the treatment and/or protection and/or modification of the appearance of the skin and/or of the hair, intended to be left on the skin and/or the hair after application.

It may for example be an after-shampoo that is intended not to be rinsed, an untangling milk, an untangling water, a smoothing water, a cuticle coat, a hairdressing product, a product for hairdressing or for redoing the hair, a sun protection product (sun cream, sun milk, sun oil), a care cream, a make-up remover, a make-up product, make-up-removing or moisturizing wipes, shaving foams, hairdressing or fixing mousses, hairdressing or fixing gels.

Shower gels, shampoos or after-shampoos, intended to be rinsed or not, comprising the polyorganosiloxane of formula (I), can thus offer improvements with respect to:
fixing of coloring carried out before or during application of the composition,
conditioning of the hair, especially on damaged hair and/or on the ends,
conditioning of the skin,
modulation of the conditioning of the hair and/or of the skin (gentle conditioning or substantial conditioning)
modulation of the conditioning of the hair and/or of the skin in relation to the proportion of nitrogen present in the polyorganosiloxane,
cosmetic effects such as softness, smoothness, untangling, shine, ease of hairdressing on dry or wetted hair,
little yellowing,
repair of sun-related damage, discoloration caused by sunlight or other external conditions, or abrasion,
preservation and/or little degradation of compounds included in the composition,
long life of an active applied on the skin and/or the hair.

The polyorganosiloxane of formula (I) can in particular be used in compositions intended for the treatment of hair that has been exposed or is exposed to dyeing compositions containing an oxidizing agent, typically compositions for permanent dyeing comprising an oxidation base or compositions for bleaching or lightening the hair, comprising an oxidizing agent. In this case it may be a shampoo, an after-shampoo, or a composition for treatment or conditioning of the hair after dyeing.

The composition according to the invention can be a hair dyeing composition. Such compositions are familiar to a person skilled in the art. It should be noted that hair dyeing compositions can be constituted of several hair dyeing products, which are intended to be mixed together by the user. In the present application, unless stated otherwise or specially specified, the term “hair dyeing composition” covers either a complete composition, or a product that is intended to be mixed with another product by the user. In the present application, the term “hair dyeing” covers any modification of hair color, whether it is coloring proper, bleaching, or a combination of bleaching and coloring.

The hair dyeing composition can include an oxidation base (oxidation dye precursor). It can include an oxidizing agent. It can include a coupling agent (dyeing modifier). It can include an agent for direct dyeing (direct dyes) The composition includes a cosmetically acceptable carrier. The composition can also include additives.

According to one embodiment, it is a permanent dyeing composition comprising an oxidation base, an oxidizing agent, and optionally a coupling agent, preferably as two products that are to be combined, one product containing the oxidation base and one product containing the oxidizing agent.

According to one embodiment, it is a composition for temporary or permanent dyeing comprising an agent for direct dyeing, and optionally an oxidizing agent.

According to one embodiment, it is a composition for bleaching or lightening the hair, comprising an oxidizing agent.

As agents for direct dyeing, we may mention neutral, acid or cationic benzene nitro dyes, neutral, acid or cationic azo direct dyes, neutral, acid or cationic quinone and especially anthraquinone direct dyes, azine direct dyes, methine direct dyes, tetraazapentamethine direct dyes, triarylmethane direct dyes, indoamine direct dyes and natural direct dyes.

As oxidizing agents, we may mention hydrogen peroxide, urea peroxide, bromates of alkali metals, persalts such as perborates and persulfates, peracids and enzymes, notably peroxidases, two-electron oxidoreductases, and four-electron oxygenases.

As coupling agents, we may mention methaphenylene-diamines, meta-aminophenols, metadiphenols, naphthalenic coupling agents and heterocyclic coupling agents.

As cosmetically acceptable carriers that are preferred in the dyeing compositions, we may mention water and/or its mixtures with solvents, for example ethanol, isopropanol, polyols and polyol ethers such as 2-butoxyethanol, propylene glycol, aromatic alcohols such as benzyl alcohol or phenoxyethanol.

The additives can be anionic, non-ionic, cationic or zwitterionic or amphoteric surfactants, anionic, neutral or cationic polymers, organic or mineral thickeners, antioxidants, penetrants, sequestering agents, perfumes, buffers, dispersants, conditioners, film-forming agents, ceramides, preservatives, opacifiers. Of course, the aforementioned ingredients can be used as additives in dyeing compositions.

The hair dyeing compositions comprising the polyorganosiloxane of formula (I) can thus:
prevent lightening of the coloring over time (fading),
promote permanence of the coloring over time,
lessen the extraction of the color, and/or
repair the hair with respect to oxidation.

In sun protection products, comprising UV filters, for example sun creams, milks, oils, and sprays, the polyorganosiloxane of formula (I) can itself have an effect of protection against the effects of UV on the skin and/or the hair. It can also have an effect of improvement of the protection provided by other agents, for example the UV filters mentioned above, against the effects of UV on the skin and/or on the hair (synergy between the polyorganosiloxane of formula (I) and other agents). The effects of UV protection can also be of benefit in terms of maintenance of the appearance or performance of the composition over time (less degradation). Thus, the polyorganosiloxane of formula (I) may prevent yellowing of the composition.

The examples presented below illustrate certain details or advantages of the invention, but are non-limiting.

### EXAMPLES

In the examples given below, the ingredient “Sil 1” is a polyorganosiloxane oil of formula (I) in which:
R is a methyl group
A is a methyl
Y is a group of formula (III)
X is a group of formula —(CH₃)₃—O—[OE]₁₀-[OP]₂
x=3
n=4
m=190.

In the following examples, the letter C indicates that it is a comparative example.

### Examples 1-4

These examples illustrate shampoos.

The amounts shown are amounts by weight of ingredient.

### Procedure

Dilute the Miracare CS (amphoteric surfactant) in water containing the Glydant with stirring. Add the silicone to the aqueous phase with stirring. Adjust the pH with citric acid (30% solution).

| Example | 1 | 2C | 3C | 4C |
|---|---|---|---|---|
| Sample | 04 MMK | 04 MMK | 04 MMK | 04 MMK |
| reference: | 006 | 006A | 006B | 006C |
| | | Control | | |
| Miracare CS, | 37.40% | 37.40% | 37.40% | 37.40% |
| Rhodia | | | | |
| Sil 1 | 0.20% | | | |
| Mirasil ADM-E | | | 1% | |
| (29% silicone | | | | |
| emulsion) | | | | |
| Mirasil DMCP 93 | | | | 0.20% |
| oil, Rhodia | | | | |
| (silicone | | | | |
| copolyol) | | | | |
| Citric acid | 0.70% | 0.70% | 0.70% | 0.70% |
| (30% solution) | | | | |
| Glydant | 0.05% | 0.05% | 0.05% | 0.05% |
| Water | 61.65% | 61.65% | 61.65% | 61.65% |
| pH | 5.20 | 5.19 | 5.15 | 5.21 |
| Viscosity (1) | 2400 | 15100 | 2520 | 5590 |
| (cP) | | | | |
| Transmittance | 99.11 | 99.15 | 99.24 | 99.22 |
| at λ = 600 nm | | | | |
| and day = 0 | | | | |
| Absorbance at λ | 0.2108 | 0.2119 | 0.2099 | 0.2109 |
| 350 nm and | | | | |
| day = 0 | | | | |
| Absorbance at λ | 0.2334 | 0.2395 | 0.2750 | 0.2504 |
| 350 nm after 21 | | | | |
| days at 45° C. | | | | |
| Delta | 0.0226 | 0.0276 | 0.0651 | 0.0395 |
| Absorbance | | | | |
| (i.e. yellowing | | | | |
| at λ 350 nm | | | | |
| after 21 days | | | | |
| at 45° C.) | | | | |

(1) Brookfield (spindle 4, 10 rev/min⁻¹25° C.)

### Test of Protection of Coloring Against Sunlight (Suntest)

Pre-wash 2 g locks of bleached platinum blond hair, 15 cm long, with a solution of sodium lauryl sulfate diluted to 15% of active substance. Pour about 900 ml of this solution into a large, 1-liter beaker. String the locks on a metal wire and immerse them in the solution for 10 minutes. Drain them a little then put them on a large inclined plate. Allow water to flow for 45 minutes. The water is at 40° C., with a flow rate of 1.80 liters per minute. After 45 minutes, untangle the locks with a comb until there are no longer any knots.

Dye the locks with a commercial l'Oréal hair coloring gel with the following references: Permanent Red Dye Fiction No. 66 (group III permanent dyeing). Rinse the locks until the water is clear, placing the ribbon on the palm of the hand. Wash each lock with 0.5 g of shampoo Mixa Bébé for 1 minute and rinse each lock for 1 minute. After draining between two fingers, dry the locks in a climate chamber at 20° C. and 50% RH for 12 hours. Treat each lock with one of the 4 shampoos (examples 1 to 4). Wash each lock with 0.5 g of shampoo for 1 minute and rinse each lock for 1 minute. After draining between two fingers, dry the locks in a climate chamber at 20° C. and 50% RH for 12 hours.

Evaluate the locks in the L, a, b system using a Dr. Lange spectral calorimeter and the software Luci 100 version 1.0 (illuminant: C; measurement geometry: d/8°; standard observer: 10°). For each lock, carry out the measurement on an area of (7*4) cm², defined beforehand. Take 6 measurements of the coefficients L, a, b on this area of lock, and take the average of the values found. The measurements are taken before and after irradiation in the Suntest.

Irradiate each treated lock for 16 hours (equivalent to 20 days of natural exposure) in a Suntest Heraeus CPS+. The Suntest provides accelerated simulation of natural exposure to UV and visible radiation. The conditions are as follows: E=500 W/m², temperature=20±5° C., relative humidity 25±5%, and the lamp used is a xenon lamp.

For each lock, measure in the L, a, b system the color of the lock obtained after treatment with the composition (examples 1 to 4) and the color of the lock obtained after the Suntest. L indicates the lightness of a color. The higher the value of L, the lighter the shade. “a” is the red-green component and “b” is the yellow-blue component. Values close to zero for a or b correspond to gray shades.

The color difference of the treated lock before and after exposure to sunlight in the Suntest reflects the deterioration of the coloring. The deterioration is calculated using the following equation:

ΔE=\sqrt{(L−Lᵢ)²+(a−aᵢ)²+(b−bᵢ)²}

ΔE represents the color difference between two locks. Lᵢ, aᵢ and bᵢ represent, respectively, the lightness, shade and saturation before irradiation. L, a, and b represent, respectively, the lightness, shade and saturation after irradiation. The test results are shown in the following table.

### Results of the Suntest:

| | Color before | | | Color after | | | |
|---|---|---|---|---|---|---|---|
| | the Suntest | | | the Suntest | | | Deterioration |
| Treatment | Lᵢ | aᵢ | bᵢ | L | a | b | of color ΔE |
| Example 3C | 20.26 | 9.82 | 7.44 | 30.15 | 12.58 | 15.66 | 13.15 |
| Example 4C | 22.24 | 11.16 | 8.41 | 32.57 | 13.43 | 17.32 | 13.83 |
| Example 2C | 23.68 | 11.1 | 9.13 | 35.32 | 13.21 | 19.12 | 15.48 |
| Example 1 | 23.47 | 11.23 | 8.73 | 29.32 | 12.58 | 16.06 | 9.47 |

It can be seen that the deterioration in color of the lock treated with the shampoo according to the invention (Example 1), after 16 hours of exposure to sunlight in the Suntest, is reduced relative to that obtained with the comparative shampoos.

### Test as Aid for Untangling the Hair (Conditioning of the Hair)

Using a coarse cutter, cut locks with width of 2.54 cm from coils of bleached platinum blond hair from the supplier International Hair Importers Inc. Prepare a solution of sodium lauryl sulfate at 15% of active substance. Pour about 900 ml of this solution into a large, 1-liter beaker. String the locks on a metal wire and immerse them in the solution for 10 minutes. Drain them a little then put them on a large inclined plate. Allow water to flow for 45 minutes. The water is at 40° C., with a flow rate of 1.80 liters per minute. After 45 minutes, untangle the locks with a comb (wide-spaced side) until there are no longer any knots, under tap water. Hanging the locks on a rack completes their pre-treatment.

The ability of the shampoos to condition the hair is determined using the Dia-Stron Miniature Tensile Tester MTT 170 and the Dia-Stron software “RHEOPC”. An adapted protocol is followed, as described in the following documents: (1) Dia-Stron MTTWIN user manual, version 1.0 (June 1995); (2) M. L. Garcia and J. Diaz, “Combability measurements on human hair”, J. Soc. Cosmet. Chem., 27, 379 (1976) and (3) Y. K. Kamath and H.-D. Weigmann, “Measurement of combing forces”, J. Soc. Cosmet. Chem., 37, 111 (1986).

In order to prepare homogeneous groups of locks, the combing force is determined for each wetted, untreated lock. Each wetted, untreated lock is combed 10 times with a comb with 4 teeth per centimeter. It is immersed in a beaker of deionized water and the excess water is removed by pinching it once between the index finger and the middle finger. This procedure causes slight tangling between the hair fibers. Each lock is combed 6 times using the combing element of the Dia-Stron MTT 170, and the mean value of the wetted, untreated combing force (F_{MNT}) is calculated. The groups of locks are constituted homogeneously using statistical analysis.

The locks are treated as follows: locks in the same group to be treated are immersed in a large beaker of tap water for one minute. The excess water is removed by pinching the lock once between the index finger and the middle finger. 0.2 ml of shampoo per 2 g of hair is deposited using a syringe. Each lock is massaged 30 times for 1 minute. The treated locks are strung on a metal wire and placed on a large inclined plate. Water is allowed to flow for 1 minute. The water is at 40° C., with a flow rate of 1.80 liters per minute. The treated locks are ready for evaluation by the “treated wet combing” test. Each treated lock is combed 6 times using the combing element of the Dia-Stron MTT 170 and the mean value of the wet, treated combing force (F_{MT}) is calculated for the groups of locks.

The difference in mean value of the combing force of the group of wetted locks before and after treatment reflects the conditioning of the hair (ease of untangling of wetted hair). The conditioning is calculated using the following equation:

ΔF=F_{MT}−F_{MNT}

ΔF represents the difference in mean value of the combing force of the group of wetted locks before and after treatment. F_{MT} represents the mean value of the combing force of the wetted, treated locks. F_{MNT} represents the mean value of the combing force of the group of wetted, untreated locks. The more negative the value of ΔF, the more effective the treatment. The test results are shown in the following table.

### Results of the Test as Aid for Untangling the Hair (Conditioning of the Hair)

| | F_{MNT} | F_{MT} | |
|---|---|---|---|
| | Mean value of the | Mean value of the | |
| | combing force of | combing force of | ΔF |
| | the group of wetted, | the group of wetted, | Conditioning |
| Treatment | untreated locks | treated locks | of the hair |
| Example 1 | 0.280 J | 0.260 J | −0.020 J |

It is found that there is conditioning of the wetted hair.

### Examples 5-9

These examples illustrate shine sprays.

The following compositions are prepared (the amounts are shown as % by weight of ingredient): Mix the Mirasil PTM and the silicone in the Mirasil CM5, with stirring. Add the ethanol, and stir the solution.

| | | Example | | | | |
|---|---|---|---|---|---|---|
| | | 5C | 6C | 7C | 8 | 9C |
| | | Reference Sample | | | | |
| | | 04 MMK | 04 MMK | 04 MMK | O4 MMK | |
| Amount | Ingredients | 003 ADME | 003 ADM | 003 DMCP 93 | 003 HALS copo | Blank |
| 5.00% | Mirasil | | | | | |
| | PTM, | | | | | |
| | Rhodia | | | | | |
| 39.00% | Mirasil | | | | | |
| | CM5, | | | | | |
| | Rhodia | | | | | |
| 1.00% | Silicone | Mirasil | Mirasil | Mirasil | Sil 1 | None |
| | | ADME, | ADM | DMCP 93, | | |
| | | Rhodia | Rhodia | Rhodia | | |
| 55.00% | Ethanol | | | | | |
| Appearance | | 2 phases | 2 phases | 1 phase | 2 phases | 1 phase |
| | | (both | (both | (turbid) | (both | (clear) |
| | | clear) | clear) | | clear) | |

Mix the Mirasil PTM and the silicone in the Mirasil CM5 with stirring. Add the ethanol and stir the solution.

### Examples 10-11

These examples illustrate sun creams.

The amounts shown are % by weight of ingredient.

| | | Example | |
|---|---|---|---|
| | | 10C | 11 |
| | | Reference Sample | |
| | Ingredients | 04 MMK 008 | 04 MMK 009 |
| Phase 1 | Brij 72, Uniqema | 2% | 2% |
| Phase 1 | Brij 721P, Uniqema | 4% | 4% |
| Phase 1 | Elefac I 205, National Starch | 4% | 4% |
| Phase 1 | Cetiol CC, Cognis | 4% | 4% |
| Phase 1 | Eusolex 2292, Merck | 7.5% | 7.5% |
| Phase 1 | Parsol 1789, Givaudan Roure | 1.5% | 1.5% |
| Phase 1 | Mirasil Wax B, Rhodia | 4% | 4% |
| Phase 1 | Mirasil CM5, Rhodia | 4% | 4% |
| Phase 1 | Carnation, Crompton | 4% | 0% |
| Phase 1 | Sil 1 | 0% | 4% |
| Phase 1 | Vitamin E Acetate | 0.3% | 0.3% |
| Phase 2 | Dermacryl 79, National Starch | 2% | 2% |
| Phase 2 | TEA 99% | 0.67% | 0.67% |
| Phase 2 | Deionized water | 10% | 10% |
| Phase 3 | Sepigel 501, Seppic | 1% | 1% |
| Phase 3 | Glycerine | 1% | 1% |
| Phase 3 | Propylene Glycol, Lyondell | 1% | 1% |
| Phase 3 | Allantoin | 0.2% | 0.2% |
| Phase 3 | Germaben II-E, ISP | 0.2% | 0.2% |
| Phase 3 | Deionized water | 35.33% | 35.33% |
| Phase 4 | Mirasun TIW 60, Rhodia | 13.3% | 13.3% |
| Phase 5 | Citric acid (50% solution) | q.s. | q.s. |
| | pH | 6.80 | 6.91 |
| | Viscosity (1) cP | 5950 | 8300 |

(1) Brookfield (spindle 6, 20 rev/min⁻¹, 25° C.)

### Procedure

1) Prepare phase 2: disperse Dermacryl in water containing TEA, with stirring.
2) Prepare phase 3: disperse Sepigel 501 in water with stirring, and homogenize, then add preservative, Allantoin, glycerine and propylene glycol.
3) Add phase 2 to phase 3.
4) Prepare phase 1: mix the ingredients, heating at 70° C., and homogenize.
5) Add phase 1 to phases 2 and 3, at the same temperature, and stir with Turrax for about 2 minutes. Add Mirasun TIW 60 with stirring. Cool the emulsion obtained to 30° C. with stirring, and adjust the pH to 6.8 with citric acid.

### Sun Cream Test

Deposit the sun cream to be analyzed at the rate of 55 to 60 mg on a TRANSPORE 3M adhesive tape. Then spread it so that the film of cream is homogeneous over the entire length of the TRANSPORE 3M adhesive tape. The sun protection factor (SPF) is measured in a climate chamber at controlled humidity. Calibrate the instrument used, Optometrics SPF 290, and carry out a blank test on TRANSPORE 3M adhesive tape. Carry out two independent measurements per sun cream (each time finding the mean value from nine measurements per TRANSPORE 3M tape). The results are presented in the following table. They show an increase in average index of protection (I.P.) for the sun cream according to the invention (Example 11).

| | Average | | | |
|---|---|---|---|---|
| | I.P. | Standard | Average | Ratio |
| Sample | In Vitro | deviation | UVA | UVA/UVB |
| Example 10C | 21.7 | 3.0 | 13.8 ± 2.6 | 0.70 ± 0.04 |
| Example 10C | 20.9 | 1.8 | 13.0 ± 1.0 | 0.70 ± 0.04 |
| Example 11 | 40.4 | 1.6 | 18.6 ± 1.1 | 0.66 ± 0.02 |
| Example 11 | 43.3 | 5.3 | 21.8 ± 2.6 | 0.68 ± 0.01 |

### Examples 12-15

These Examples illustrate cuticle coats.

The amounts are shown as wt. % of ingredient.

| | | Example | | | |
|---|---|---|---|---|---|
| | | 12C | 13C | 14C | 15 |
| | | Reference Sample | | | |
| | | 04 MMK 019 | 04 MMK 019 | 04 MMK 019 | 04 MMK 019 |
| amounts | Ingredients | Control | ADM | DMCP 93 | HALS copolyol |
| 2.00% | Mirasil PTM, | | | | |
| | Rhodia | | | | |
| 16.00% | Mirasil CM5, | | | | |
| | Rhodia | | | | |
| 2.00% | Silicone | None | Mirasil ADM, | Mirasil | Sil 1 |
| | | | Rhodia | DMCP 93, | |
| | | | | Rhodia | |
| 80.00% | Mirasil C-DML, | | | | |
| | Rhodia | | | | |
| appearance | | 1 phase | 1 phase | 1 phase | 1 phase |
| | | (clear, | (clear, | (clear, | (clear, |
| | | viscous) | viscous) | viscous) | viscous) |

Mix the Mirasil PTM and the silicone in the Mirasil CM5, with stirring. Add the Mirasil C-DML and mix the silicone oils together.

1-23. (canceled)

24. A cosmetic composition comprising a cosmetically acceptable carrier and a polyorganosiloxane, said polyorganosiloxane having the following general formula (I):
[RₐX_{b}Y_{c}SiO_{(4-a-b-c)/2}]_{N} (I)
wherein:
RₐX_{b}Y_{c}SiO_{(4-a-b-c)/2}, which are identical or different, represent linear or branched units in the polyorganosiloxane,
N represents the number of silicon atoms in the polyorganosiloxane, greater than or equal to 3,
a, b and c, which are identical or different, are numbers equal to 0, 1, 2 or 3,
a+b+c, which is identical or different, is equal to 0, 1, 2 or 3,
R, which is identical or different, represents a C₁-C₁₈ alkyl group, a C₆-C₁₂ aryl group, a C₆-C₁₂ aralkyl group, a C₆-C₁₂ alkaryl group, a group of formula —[CH₂]₃—NH[CH₂]₂—NH₂, a group of formula —[CH₂]₃—NH₂, a hydroxyl group, a C₁-C₁₈ alkoxy group, a C₁-C₁₈ hydroxyalkyl group, or a C₁-C₁₈ hydroxyalkyletheralkyl group, said groups being optionally substituted,
X, which is identical or different, represents a polyalkoxy group carried by a silicon atom,
Y, which is identical or different, represents a group carried by a silicon atom, of formula —R⁴—U-Hals, in which:
R⁴ is a divalent hydrocarbon binding group, optionally an alkyl group,
U is a covalent bond or a divalent swivel group comprising a heteroatom,
Hals is a functional group comprising a sterically hindered amine,
at least one silicon atom of the polyorganosiloxane bears a group X, and
at least one silicon atom of the polyorganosiloxane bears a group Y.

25. The composition as claimed in claim 24, wherein the groups X and Y are carried by different silicon atoms.

26. The composition as claimed in claim 24, wherein the group X has the formula L^{x}Z^{x}-Palk, in which:
L^{x} is a divalent bonding group, optionally an alkyl group,
Z^{x} is a covalent bond or a divalent connecting group comprising a heteroatom,
Palk is a group of formula [OE]ₛ-[OP]ₜ—X′, in which
OE is a group of formula —CH₂—CH₂—O—,
OP is a group of formula —CH₂—CHCH₃—O— or —CHCH₃—CH₂—O—,
X′ is a hydrogen atom or a hydrocarbon-based group,
s is an average number greater than 1, and
t is an average number greater than or equal to 0.

27. The composition as claimed in claim 24, wherein X is a group of formula —CH₂—CH₂—CH₂—O—[OE]_{q}-[OP]ᵣ—X′, in which:
q≧5,
r≧0, and
x′ is a hydrogen atom.

28. The composition as claimed in claim 24, wherein group Y has the following formula (II): 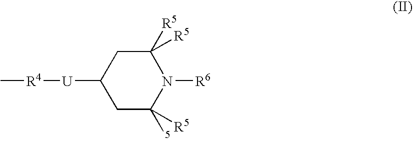
in which:
R⁴, which is identical or different, is a divalent hydrocarbon divalent selected from:
linear or branched alkylene groups having 2 to 18 carbon atoms;
alkylene-carbonyl groups, the linear or branched alkylene portion of which has 2 to 20 carbon atoms;
alkylene-cyclohexylene groups, the linear or branched alkylene portion of which has 2 to 12 carbon atoms and the cyclohexylene portion has an —OH group and optionally 1 or 2 alkyl groups having 1 to 4 carbon atoms;
groups of formula —R⁷—O—R⁷ in which the R⁷ groups, which are identical or different, represent alkylene groups having 1 to 12 carbon atoms;
groups of formula R⁷—O—R⁷ in which the R⁷ groups have the meanings stated previously and one of them or both of them are substituted with one or two —OH groups;
groups of formula —R⁷—COO—R⁷ in which the R⁷ groups have the meanings stated previously; and
groups of formula —R⁸—O—R⁹—O—CO—R⁸ in which the R⁸ and R⁹ groups, which are identical or different, represent alkylene groups having 2 to 12 carbon atoms and the R⁹ group is optionally substituted with a hydroxyl group;
U represents —O— or —NR¹⁰—, R¹⁰ being a hydrogen atom, a linear or branched alkyl group comprising 1 to 6 carbon atoms;
R⁵, which is identical or different, is a linear or branched alkyl group having 1 to 3 carbon atoms or a phenyl group; and
R⁶ represents a hydrogen atom, an R⁵ group or a free radical O.

29. The composition as claimed in claim 24, wherein group Y is a group of the following formula (III):

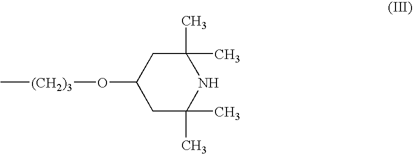

30. The composition as claimed in claim 24, wherein group R, which is identical or different, is a methyl, ethyl, isopropyl, tert-butyl, n-hexyl, octyl, trifluoropropyl, or phenyl group.

31. The composition as claimed in claim 24, wherein R is a methyl group.

32. The composition as claimed in claim 24, wherein the polyorganosiloxane of formula (I) is a linear polyorganosiloxane.

33. The composition as claimed in claim 24, wherein the polyorganosiloxane of formula (I) has the following formula (IV):
A-SiR₂—O—[SiRXO]ₓ—[SiRYO]ₙ—[SiR₂O]ₘ—O—SiR₂-A (IV)
in which:
R, which is identical or different, is a group as above defined,
X, which is identical or different, is a group as above defined,
Y, which is identical or different, is a group as above defined,
A, which is identical or different, is a group R, Y, or X,
x is an average number greater than 0,
n is an average number greater than 0,
m is an average number greater than 0, and
x+m+n+2=N.

34. The composition as claimed in claim 33, wherein:
A is a group R, and
R is a methyl group.

35. The composition as claimed in claim 33, wherein:
m≧10, optionally m≧100,
x≧1, and
n≧3.

36. The composition as claimed in claim 33, wherein the polyorganosiloxane of formula (I) has between 0.1 and 0.5 wt % of nitrogen atoms.

37. The composition as claimed in claim 33, wherein:
m≧50, and
0.00011≦n/(m+n+x)≦0.5, optionally 0.005≦n/(m+n+x)≦0.1.

38. The composition as claimed in claim 33, wherein:
m≧50, and
0.001≦x(m+n+x)≦0.5, optionally 0.01≦x/(m+n+x)≦0.2.

39. The composition as claimed in claim 24, wherein the polyorganosiloxane of formula (I) has a viscosity between 1000 and 1000000 mPa·s, optionally between 10000 and 100000 mPa·s.

40. The composition as claimed in claim 24, wherein the polyorganosiloxane is present in the composition in the form of an emulsion of droplets comprising the polyorganosiloxane of formula (I), dispersed in a cosmetically acceptable aqueous carrier.

41. The composition as claimed in claim 24, wherein:
the emulsion is an emulsion with an average droplet size greater than or equal to 0.15 μm, obtained using an emulsifier of the polyorganosiloxane of formula (I), optionally in a proportion less than 10 wt. % relative to the amount of polyorganosiloxane of formula (I), or
the emulsion is a microemulsion with an average droplet size less than 0.15 μm, obtained using an emulsifier of the polyorganosiloxane of formula (I), optionally in a proportion greater than 10 wt. % relative to the amount of polyorganosiloxane of formula (I).

42. The composition as claimed in claim 24, further comprising, in addition to the polyorganosiloxane of formula (I):
at least one anionic and/or amphoteric surfactant, alone or mixed,
optionally, at least one stabilizer and/or conditioner and/or conditioning aid,
optionally another polyorganosiloxane, and
optionally a UV filter.

43. The composition as claimed in claim 42, wherein the stabilizer and/or conditioner and/or conditioning aid is a cationic polymers derived from polysaccharides,
synthetic cationic polymers,
a crosslinked polyacrylate, or
glycol distearate (EGDS),

44. The composition as claimed in claim 24, being a cosmetic composition for care of the skin and/or of the hair, optionally for cleaning and/or treatment of the skin and/or of the hair, said composition being in the form of a fluid.

45. The composition as claimed in claim 24, being a shower gel, a shampoo, an after-shampoo, intended to be rinsed or not.

46. The composition as claimed in claim 24, being a sun protection product.
